(19) United States
(12) Patent Application Publication
(10) Pub. No.: US 2014/0017752 A1
(43) Pub. Date: Jan. 16, 2014

(54) PROCESS FOR COMPLETE ANAEROBIC DIGESTION OF POLYMER MIXTURES

(71) Applicant: BASF SE, Ludwigshafen (DE)

(72) Inventors: Carsten Sinkel, Mannheim (DE); Robert Loos, Ludwigshafen (DE); Karlheinz Jochem, Lingenfeld (DE); Kai Oliver Siegenthaler, Mannheim (DE); Xin Yang, Bensheim (DE); Ulf Küper, Mannheim (DE); Mathias Zimmermann, Worms (DE)

(21) Appl. No.: 13/937,753

(22) Filed: Jul. 9, 2013

Related U.S. Application Data
(60) Provisional application No. 61/669,692, filed on Jul. 10, 2012.

(51) Int. Cl. C12P 5/02

(52) U.S. Cl. CPC C12P 5/023
USPC 435/167

(57) ABSTRACT

The present invention relates to a process for complete anaerobic digestion of polymer mixtures comprising:
a) 25 to 95% by weight of a polyhydroxyalkanoate selected from the group consisting of poly-4-hydroxybutyrates, poly-3-hydroxybutyrates, poly(3-hydroxybutyrate-co-3-hydroxyvalerates), poly(3-hydroxybutyrate-co-3-hydroxyhexanoates) and poly(3-hydroxybutyrate-co-4-hydroxybutyrates), and
b) 5 to 75% by weight of an aliphatic-aromatic polyester comprising:
   i) 65 to 95 mol %, based on components i to ii, of one or more C₅-C₃₆-dicarboxylic acid derivatives or C₅-C₃₆-dicarboxylic acids;
   ii) 35 to 5 mol %, based on components i to ii, of a terephthalic acid derivative or of a terephthalic acid;
   iii) 98 to 100 mol %, based on components i to ii, of an C₂-C₈-alkylenediol or C₂-C₆-oxyalkylenediol;
   iv) 0 to 2% by weight, based on components i to iii, of at least one polyfunctional compound comprising at least two isocyanate, isocyanurate, oxazoline or epoxide groups or at least three alcohol or carboxylic acid groups.

The present invention relates to a process for complete anaerobic digestion of polymer mixtures comprising:
a) 25 to 95% by weight of a polyhydroxyalkanoate selected from the group consisting of poly-4-hydroxy-butyrates, poly-3-hydroxybutyrates, poly(3-hydroxy-butyrate-co-3-hydroxyvalerate), poly(3-hydroxybutyrate-co-3-hydroxyhexanoates) and poly(3-hydroxybutyrate-co-4-hydroxybutyrates), and
b) 5 to 75% by weight of an aliphatic-aromatic polyester comprising:
   i) 65 to 95 mol %, based on components i to ii, of one or more C₅-C₃₆-dicarboxylic acid derivatives or C₅-C₃₆-dicarboxylic acids;
   ii) 35 to 5 mol %, based on components i to ii, of a terephthalic acid derivative or of a terephthalic acid;
   iii) 98 to 100 mol %, based on components i to ii, of an C₂-C₈-alkylenediol or C₂-C₆-oxyalkylenediol;
   iv) 0 to 2% by weight, based on components i to iii, of at least one polyfunctional compound comprising at least two isocyanate, isocyanurate, oxazoline or epoxide groups or at least three alcohol or carboxylic acid groups.

WO-A 92/09654 describes linear aliphatic-aromatic polyesters which are biodegradable. Crosslinked biodegradable polyesters are described in WO-A 96/15173. The polyesters described in WO-A 92/09654 and WO-A 96/15173, in mixtures with polyhydroxyalkanoates, however, do not have an anaerobic digestion rate significantly exceeding the calculated polyhydroxyalkanoate content.

U.S. Pat. No. 5,281,691 and US 2004/0225269 describe mixtures of polyhydroxyalkanoates and aliphatic-aromatic polyesters having a very unspecific terephthalic acid content. Anaerobic digestion rates for these mixtures are not described; more particularly, there is no indication in these documents that the aliphatic-aromatic polyester itself can also likewise be anaerobically digested if the terephthalic acid content is appropriately small and the correct mixing ratio with polyhydroxyalkanoate is selected.

Films consisting exclusively of polymer component a (polyhydroxyalkanoate) are anaerobically digestible, but are not convincing in terms of their mechanical properties and their processibility.

The aim of the present invention was accordingly to provide a process for producing mechanically durable films which additionally have good anaerobic biodegradability and good processibility.

Interestingly, the inventive mixtures wherein the polymer component b has a relatively low terephthalic acid content have an enhanced anaerobic digestion rate which far exceeds the calculated value for the polyhydroxyalkanoate content. This is surprising and suggests that the inventive polymer mixtures have synergism with regard to anaerobic digestion.

By using the polyesters described at the outset, these having a narrowly defined terephthalic acid content and a narrowly defined content of a polyfunctional component iv, it was surprisingly possible to produce mechanically durable films with a high anaerobic digestion rate.

The invention is described in detail hereinafter.

Polyhydroxyalkanoates (polymer component a) are primarily understood to mean poly-4-hydroxybutyrates and poly-3-hydroxybutyrates or poly-3-hydroxybutyrate-co-4-hydroxybutyrates, and also copolyesters of the aforementioned polyhydroxybutyrates with 3-hydroxyvalerate, 3-hydroxyhexanoate and/or 3-hydroxyoctanoate. Poly-3-hydroxybutyrates are sold, for example, by PHB Industrial under the Biocycle® brand name and by Tianan under the Enmat® name.

Poly-3-hydroxybutyrate-co-4-hydroxybutyrates are known, particularly from Metabolix. They are sold under the Mirel® trade name.

Poly-3-hydroxybutyrate-co-3-hydroxyhexanoates are known, for example, from Kaneka. Poly-3-hydroxybutyrate-co-3-hydroxyhexanoates generally have a 3-hydroxyhexanoate content of 1 to 20 and preferably of 3 to 15 mol % based on the butyrate content.

Synergism in the course of anaerobic digestion is found for all aforementioned polyhydroxyalkanoates. It is particularly marked in the case of the copolymers: poly-3-hydroxybutyrate-co-3-hydroxyvalerate and especially in the case of poly-3-hydroxybutyrate-co-4-hydroxybutyrate and poly-3-hydroxybutyrate-co-3-hydroxyhexanoate.

The aforementioned copolymers are particularly preferred for the inventive polymer mixtures.

The polyhydroxyalkanoates generally have a molecular weight Mw of 100 000 to 1 000 000 and preferably of 300 000 to 600 000.

The polyhydroxyalkanoates are preferably produced by fermentation, as described, for example, in WO-A 2008010296 or WO-A 1999064498.

The polyester component b) is generally synthesized in a two-stage reaction cascade (see WO09/127,555 and WO09/127,556). First of all, the dicarboxylic acid derivatives, as in the synthesis examples, are reacted together with the diol (for example 1,4-butanediol) in the presence of a transesterification catalyst to give a prepolyester. Subsequently, the melt of the prepolyester thus obtained is typically condensed up to the desired viscosity with distillative removal of diol released under reduced pressure at an internal temperature of 200 to 250° C. within 3 to 6 hours. The catalysts used are typically zinc catalysts, aluminum catalysts and especially titanium catalysts. Titanium catalysts such as tetraisopropyl orthotitanate and especially tetrabutyl orthotitanate (TBOT) have the advantage over the tin, antimony, cobalt and lead catalysts frequently used in the literature, for example tin dioctanoate, that residual amounts of the catalyst or conversion product of the catalyst remaining in the product are less toxic. This fact is particularly important in the case of the biodegradable polyesters, since they get directly into the environment, for example, in the form of composting bags or mulch films.

The inventive polyesters are optionally subsequently chain-extended by the methods described in WO 96/15173 and EP-A 488 617. The prepolyester is reacted, for example, with chain extenders vib), such as with diisocyanates or with epoxide-containing polymethacrylates, in a chain extension reaction to give a polyester having a VN of 60 to 450 ml/g, preferably 80 to 250 ml/g.

More preferably, the polyester component b) is prepared by the continuous process described in WO2009127556. The abovementioned viscosity number ranges serve merely as indications for preferred process variants, but should not be considered to be restrictive of the subject matter of the present application.

As well as the above-described continuous process, the inventive polyesters can also be prepared in a batch process. For this purpose, the aliphatic and aromatic dicarboxylic acid derivatives, the diol and a branching agent iva are mixed in any metering sequence and condensed to give a prepolyester. Optionally, with the aid of a chain extender, a polyester with the desired viscosity number can be prepared.

With the abovementioned processes, it is possible to obtain, for example, polybutylene terephthalate succinates, azelates, brassylates and especially adipates and sebacates having an acid number measured to DIN EN 12634 of less than 1.0 mg KOH/g and a viscosity number of greater than 130 ml/g, and an MVR to ISO 1133 of less than 6 cm³/10 min (190° C., weight 2.16 kg).

For other applications, inventive polyesters with higher MVR to ISO 1133 of up to 30 cm³/10 min (190° C., weight 2.16 kg) may be of interest. The polyesters generally have an MVR to ISO 1133 of 1 to 30 cm³/10 min and preferably 2 to 20 cm³/10 min (190° C., weight 2.16 kg).

Sebacic acid, azelaic acid and brassylic acid (i) are obtainable from renewable raw materials, especially from vegetable oils, for example castor oil.

Terephthalic acid ii is used in 5 to 35 mol % and preferably 10 to 25 mol % based on the diacid components i and ii.

Terephthalic acid and the aliphatic dicarboxylic acid can be used either as the free acid or in the form of ester-forming derivatives. Ester-forming derivatives are especially the di-C₁— to —C₆-alkyl esters, such as dimethyl, diethyl, di-n-propyl, diisopropyl, di-n-butyl, diisobutyl, di-t-butyl, di-n-pentyl, diisopentyl or di-n-hexyl esters. Anhydrides of the dicarboxylic acids can likewise be used.

The dicarboxylic acids or their ester-forming derivatives can in this case be used individually or as a mixture.

1,4-Butanediol is likewise obtainable from renewable raw materials. WO 09/024,294 discloses a biotechnological method for production of 1,4-butanediol, proceeding from different carbohydrates with microorganisms from the class of the Pasteurellaceae. Succinic acid is likewise obtainable by means of biotechnological methods.

In general, on commencement of the polymerization, the diol (component iii) is used relative to the acids (components i and ii) in a ratio of diol to diacids of 1.0 to 2.5:1 and preferably 1.3 to 2.2:1. Excess amounts of diol are drawn off during the polymerization, such that an approximately equimolar ratio is established at the end of the polymerization. “Approximately equimolar” is understood to mean a diol/diacids ratio of 0.98 to 1:1.

The polyesters mentioned may have hydroxyl and/or carboxyl end groups in any desired ratio. The semiaromatic polyesters mentioned may also have end group modification. For example, OH end groups may be acid-modified by reaction with phthalic acid, phthalic anhydride, trimellitic acid, trimellitic anhydride, pyromellitic acid or pyromellitic anhydride. Preference is given to polyesters having acid numbers less than 1.5 mg KOH/g.

In general, a branching agent iva and optionally additionally a chain extender ivb selected from the group consisting of: a polyfunctional isocyanate, isocyanurate, oxazoline, epoxide, carboxylic anhydride, an at least trifunctional alcohol or an at least trifunctional carboxylic acid are used. Useful chain extenders ivb include polyfunctional and especially difunctional isocyanates, isocyanurates, oxazolines, carboxylic anhydride or epoxides. The crosslinkers iva) are generally used in a concentration of 0 to 2% by weight, preferably 0.07 to 1% by weight and especially preferably 0.1 to 0.5% by weight, based on the polymer obtainable from components i to iii. The chain extenders ivb) are generally used in a concentration of 0 to 2% by weight, preferably 0.1 to 1% by weight and especially preferably 0.35 to 1% by weight, based on the total weight of components i to iii.

Chain extenders and alcohols or carboxylic acid derivatives having at least three functional groups can also be regarded as branching agents. Particularly preferred compounds have three to six functional groups. Examples include: tartaric acid, citric acid, malic acid; trimethylolpropane, trimethylolethane; pentaerythritol; polyether triols and glycerol, trimesic acid, trimellitic acid, trimellitic anhydride, pyromellitic acid and pyromellitic dianhydride. Preference is given to polyols such as trimethylolpropane, pentaerythritol and especially glycerol. By means of the components iv, it is possible to form biodegradable polyesters having structural viscosity. The rheological characteristics of the melts are improved; the biodegradable polyesters can be processed more easily, for example better drawn to films by melt solidification.

In general, it is advisable to add the crosslinking (at least trifunctional) compounds at a comparatively early point in the polymerization.

Suitable bifunctional chain extenders are the following compounds:

An aromatic diisocyanate ivb is understood to mean particularly tolylene 2,4-diisocyanate, tolylene 2,6-diisocyanate, diphenylmethane 2,2′-diisocyanate, diphenylmethane 2,4′-diisocyanate, diphenylmethane 4,4′-diisocyanate, naphthylene 1,5-diisocyanate or xylylene diisocyanate. Among these, diphenylmethane 2,2′-, 2,4′- and 4,4′-diisocyanate are particularly preferred. In general, the latter diisocyanates are used as a mixture. In minor amounts, for example up to 5% by weight, based on the total weight, the diisocyanates may also comprise uretdione groups, for example for capping of the isocyanate groups.

An aliphatic diisocyanate in the context of the present invention is understood particularly to mean linear or branched alkylene diisocyanates or cycloalkylene diisocyanates having 2 to 20 carbon atoms, preferably 3 to 12 carbon atoms, e.g. hexamethylene 1,6-diisocyanate, isophorone diisocyanate or methylenebis(4-isocyanatocyclohexane). Particularly preferred aliphatic diisocyanates are isophorone diisocyanate and especially hexamethylene 1,6-diisocyanate.

The inventive polyesters generally have a number-average molecular weight (Mn) in the range from 5000 to 100 000, especially in the range from 10 000 to 60 000 g/mol, preferably in the range from 15 000 to 38 000 g/mol, a weight-average molecular weight (Mw) from 30 000 to 300 000, preferably 35 000 to 200 000 g/mol, and an Mw/Mn ratio of 1 to 6, preferably 2 to 4. The viscosity number is between 30 and 450, preferably from 50 to 400 ml/g and especially preferably from 80 to 250 ml/g (measured in o-dichlorobenzene/phenol (weight ratio 50/50)). The melting point is in the range from 30 to 100, preferably in the range from 35 to 80° C.

Preference is given to biodegradable polyesters having the following constituents:

Component i), the aliphatic dicarboxylic acid, is preferably adipic acid and/or sebacic acid.

Component iii), the diol, is preferably 1,4-butanediol.

Component iv), the branching agent iva, is preferably glycerol.

The inventive polymer mixtures comprise 25 to 95% by weight, preferably 30 to 90% by weight and especially preferably 35 to 85% by weight of polyhydroxyalkanoate (a) and accordingly 5 to 75% by weight, preferably 10 to 70% by weight and especially preferably 15 to 65% by weight of polyester component b.

In a preferred embodiment, 1 to 50% by weight, based on the total weight of the film (components a to c), of an organic filler c) selected from the group consisting of: native or plasticized starch, natural fibers, wood flour, comminuted cork, ground bark, nutshells, ground presscake (vegetable oil refinery), dry production residues from the fermentation or distillation of drinks, for example beer, brewed lemonades (e.g. Bionade), wine or sake, and/or of an inorganic filler selected from the group consisting of: chalk, graphite, gypsum, conductive black, iron oxide, calcium chloride, dolomite, kaolin, silica (quartz), sodium carbonate, titanium dioxide, silicate, wollastonite, mica, montmorillonite, talc, glass fibers and mineral fibers.

Starch and amylose may be native, i.e. non-thermoplasticized, or may have been thermoplasticized with plasticizers, for example glycerol or sorbitol (EP-A 539 541, EP-A 575 349, EP 652 910). Thermoplasticized starch is especially preferred because it is itself likewise anaerobically digested, and films comprising polymer components a and b in addition to starch give good mechanical values. Surprisingly, mixtures of starch and polymer component b (without polymer component a) do not exhibit any synergism in terms of anaerobic digestibility. If starch is added to the polymer mixture of a and b, in addition to the digestion of the starch, the above-described synergistic anaerobic digestion characteristics are also found. The thermoplasticized starch is added to the polymer mixtures comprising components a and b generally in a ratio of 0 to 50, preferably 5 to 50 and especially preferably 10 to 35% by weight. Films produced therefrom have outstanding tear propagation resistance and, at the same time, full anaerobic digestion. They are especially suitable for production of very thin films with tear propagation resistance.

Natural fibers are understood for example to mean cellulose fibers, hemp fibers, sisal, kenaf, jute, flax, abacca, coconut fibers, or else regenerated cellulose fibers (rayon) such as Cordenka fibers.

Addition of mineral fillers, such as chalk, graphite, gypsum, conductive black, iron oxide, calcium chloride, dolomite, kaolin, silica (quartz), sodium carbonate, titanium dioxide, silicate, wollastonite, mica, montmorillonite or talc, can significantly improve the mechanical properties of the films, for example tear propagation resistance. In general, the mineral fillers are used in a concentration of 1 to 50%, preferably 4 to 30% and especially preferably 8 to 25% by weight, based on the polymer components i to iv.

The anaerobically digestible polyester mixtures a,b may comprise further polymers such as polylactic acid, polycaprolactone, aliphatic polyesters, polyglycolic acid and polypropylene carbonate in an amount of 0 to 30% by weight, preferably 5 to 20% by weight. Aliphatic polyesters are understood to mean polyesters formed from aliphatic C₂-C₁₂-alkanediols and aliphatic C₄-C₃₆-alkanedicarboxylic acids such as polybutylene succinate (PBS), polybutylene adipate (PBA), polybutylene succinate adipate (PBSA), polybutylene succinate sebacate (PBSSe), polybutylene sebacate adipate (PBSeA), polybutylene sebacate (PBSe) or corresponding polyester amides. The aliphatic polyesters are marketed by Showa Highpolymers under the Bionolle® name and by Mitsubishi under the GSPIa® name. More recent developments are described in WO 2010/034711.

The biodegradable polyester mixtures may comprise further ingredients which are known to those skilled in the art but are not essential to the invention. For example, the additives customary in the plastics industry, such as stabilizers; nucleating agents; neutralizing agents; lubricants and release agents such as stearates (especially calcium stearate) or erucamide or behenamide; plasticizers, for example citric esters (especially acetyl tributyl citrate), glyceryl esters such as triacetylglycerol or ethylene glycol derivatives, surfactants such as polysorbates, palmitates or laurates; waxes, for example beeswax or beeswax esters; antistats, UV absorbers; UV stabilizers; antifogging agents or dyes. The additives are used in concentrations of 0 to 5% by weight, especially 0.1 to 2% by weight, based on the inventive polyesters. Plasticizers may be present in the inventive polyesters in 0.1 to 10% by weight.

The inventive biodegradable polyester mixtures can be produced from the individual components by known methods (EP 792 309 and U.S. Pat. No. 5,883,199). For example, all mixing partners can be mixed and reacted in one process step in the mixing apparatus known to those skilled in the art, for example kneaders or extruders, at elevated temperatures, for example from 120° C. to 250° C.

The polymer mixtures themselves may comprise 0.05 to 2% by weight of a compatibilizer. Preferred compatibilizers are carboxylic anhydrides such as maleic anhydride and especially the above-described copolymers containing epoxide groups and based on styrene, acrylic esters and/or methacrylic esters. The units bearing epoxide groups are preferably glycidyl (meth)acrylates. Copolymers containing epoxide groups of the abovementioned type are sold, for example, by BASF Resins B.V. under the Joncryl® ADR brand. One example of a particularly suitable compatibilizer is Joncryl® ADR 4368.

Component iv, the aforementioned fillers or the other aforementioned assistants are preferably added to polymer component a or b through previously produced masterbatches of the assistants.

The process of degradation of polymers is explained in detail hereinafter, and the differences between abiotic, aerobic and anaerobic digestion are discussed in detail.

In general, polymers or polymer mixtures may be subject to a degradation process in two fundamentally different ways. First, the polymeric structure of a macromolecule can be broken up exclusively under the influence of abiotic factors (physicochemical parameters, for example: UV radiation, temperature, pH, humidity, influence of reactive oxygen species), which ultimately leads to conversion of the polymer to oligomers, monomers or reaction products resulting from the degradation. This contrasts with the biodegradation of polymers, which is based primarily on the biochemical interaction of microorganisms (bacteria, archaea, fungi) with the polymer. The breaking of the chemical bonds in the polymer is achieved here by specific interactions with the enzymes of the microorganisms. The interplay of a wide variety of different microorganisms and enzymes thereof finally leads to mineralization of the polymer. Mineralization does not just convert the polymer back to monomers or oligomers, but converts it enzymatically to the microbial metabolic end products water, carbon dioxide and methane (under anaerobic conditions). Abiotic degradation and biodegradation frequently also proceed in parallel—what is crucial, however, is that mineralization is at the end of the biodegradation.

Both the biodegradation and the physicochemical degradation of polymers lead to a loss of the characteristic polymer properties.

The biodegradation of macromolecules per se is a very diverse process which results in different degradation rates in relation to the habitat and the abiotic parameters prevailing therein. As well as the abiotic boundary conditions, efficient biodegradation also requires correspondingly high compatibility between polymer and enzyme. Consequently, a high degradation rate can be achieved when the conditions prevailing in the habitat are optimal for the microorganisms involved and a specific interaction between polymer chain and enzyme is ensured. Crucial factors here are the temperature, the pH, the presence or absence of oxygen and the availability of nutrients, minerals and trace elements. According to the combination of these factors, the corresponding habitat is dominated by different consortia with a very variable number of microorganisms (total cell count: cells per unit volume; species diversity: number of microbial species in the habitat), and these lead to the different degradation rates described.

For the biodegradation of synthetic polymers, particular interest attaches to the “ecological systems”, which find use in the context of biological waste treatment. As well as composting and the biological treatment of wastewater, particular mention should also be made of biogas-forming degradation under anaerobic conditions in biogas plants. In this context, as well as the metabolic end products of aerobic digestion (H₂O and CO₂), methane is additionally formed, and this can be utilized later for generation of electrical power or be fed into the natural gas grid as biomethane. The process of anaerobic digestion (AD) is a complex multistage microbial reaction cascade (hydrolysis→acidogenesis→acetogenesis→methanogenesis), which combines the conversion of the polymers to monomers and the subsequent metabolic reactions of the intermediates extending as far as H₂O, CO₂ and CH₄. It is important here to mention that this process is conducted not by an individual, independent microorganism, but by a multitude of microorganisms each responsible for a corresponding component step of the reaction cascade. On the industrial scale, the process takes place either in plants for dry fermentation (dry matter >20-40% (w/w)) or for wet fermentation (dry matter <12-15% (w/w)). While wet plants are currently being used in Germany principally by farmers for biogas production from manure or renewable raw materials, plants for dry fermentation are also finding use in the elimination of organic waste in waste management. In the case of dry fermentation, a distinction can in turn be made between the continuously operated plug flow plants (continuous process; dry matter >20-30% (w/w)) and the discontinuously operated box fermenters (batch process; dry matter >30-40% (w/w)). This is just a selection of the available technologies. The efficiency of the overall process is based on how much biogas (CO₂ and CH₄ volume) can be obtained from the amount of substrate (carbon source) supplied and on the quality of the resulting biogas (CH₄ content). The methane formation potential of a substrate can thus be determined via the measurement of the methane formed within a defined unit of time and compared quantitatively with other substrates. For simplification and reproducibility of the method, a simple volume determination of the biogas formed is often conducted, in which the CO₂ is scrubbed out by means of sodium hydroxide solution beforehand. It is thus possible to determine the volume of methane formed by a direct route. Alternatively, it is also possible first to determine the total volume of the biogas, followed by the quantitative analysis of the biogas composition by means of a gas chromatograph. In view of later industrial use and better comparability, anaerobic digestion will generally be considered over a period of not more than 2 months in all test methods.

Methods already described for determination of the biodegradability of polymers and other chemical substances can be found, for example, in the following ISO test methods:

ISO 11734
Water quality—Evaluation of the “ultimate” anaerobic biodegradability of organic compounds in digested sludge—Method by measurement of the biogas production

ISO 15985
Plastics—Determination of the ultimate anaerobic biodegradation and disintegration under high-solids anaerobic-digestion conditions—Method by analysis of released biogas

ISO 14853
Plastics—Determination of the ultimate anaerobic biodegradation of plastic materials in an aqueous medium—Method by analysis of released biogas

### or in the VDI Guideline

VDI 4630
Fermentation of organic materials Characterisation of the substrate, sampling, collection of material data, fermentation tests—the VDI guideline provides standardized rules and specifications for the conduct of fermentation experiments; this guideline makes it possible for the first time to achieve comparable, representative experimental results. The fermentation experiments detailed in the experimental section were therefore designed in analogy to VDI 4630.

Complete anaerobic fermentation of the inventive polymer mixtures was evidenced especially by the methods according to ISO 15985 and VDI 4630. The present process shall also comprise test methods which derive from the measurement principle, the underlying microorganisms and the concentrations of the microorganisms used in the two abovementioned test methods. Because of the ease of reproducibility and the usefulness of the results, the method according to VDI 4630 is very particularly preferred. The term “anaerobic digestibility” used in the present application is thus based primarily on VDI 4630.

The present invention accordingly relates more particularly to a process for complete anaerobic digestion of polymer mixtures of the composition:
a) 25 to 95% by weight, based on components a and b, of a polyhydroxyalkanoate selected from the group consisting of poly-4-hydroxybutyrates, poly-3-hydroxybutyrates, poly(3-hydroxybutyrate-co-3-hydroxyvalerates), poly(3-hydroxybutyrate-co-3-hydroxyhexanoates) and poly(3-hydroxybutyrate-co-4-hydroxybutyrates), and
b) 5 to 75% by weight, based on components a and b, of an aliphatic-aromatic polyester comprising:
   i) 65 to 95 mol %, based on components i to ii, of one or more C₅-C₃₆-dicarboxylic acid derivatives or C₅-C₃₆-dicarboxylic acids;
   ii) 35 to 5 mol %, based on components i to ii, of a terephthalic acid derivative or of a terephthalic acid;
   iii) 98 to 100 mol %, based on components i to ii, of an C₂-C₈-alkylenediol or C₂-C₆-oxyalkylenediol;
   iv) 0 to 2% by weight, based on components i to iii, of at least one polyfunctional compound comprising at least two isocyanate, isocyanurate, oxazoline or epoxide groups or at least three alcohol or carboxylic acid groups;
      wherein the anaerobic digestion can be determined by means of one of the processes according to VDI 4630 or ISO 15985. As mentioned above, the method according to VDI 4630 is particularly preferred.

The inoculation material used in VDI 4630 was an LUFA sludge. The content of dry matter was 3.7% of the fresh matter, and the ash made up 1.8% of the fresh matter (49.5% of the dry matter) and the organic matter (calcination loss) 1.9% of the fresh matter (50.5% of the dry matter); the pH was about 7.4 to 7.8.

The aforementioned complete anaerobic digestion of said polymer mixtures is understood to mean that not just mixture component a (polyhydroxyalkanoate) is degraded, which is already known from the literature, but also the aliphatic-aromatic polyester b.

Complete anaerobic digestion is understood to mean a digestion rate (biogas evolution measured to VDI 4630 in 42 days) of the polymer mixture a+b, based on the polymer component a, of greater than 70%. Examples of this are given in table 1.

The complete anaerobic digestion of polymer mixtures having a high content of polymer component a of greater than 70% and especially greater than 90% is determined as follows. It is assumed that the proportion of polymer component a has been 100% degraded. The amount of biogas determined experimentally for this purpose is subtracted from the amount of biogas formed, and the excess is ascribed to the digestion of polymer component b. This value can be used to check, on the basis of values tabulated above, whether component b has been degraded to an extent of greater than 90%.

In the case of polymer mixtures comprising 80% by weight or more of polymer component b, based on components a and b, are not completely degraded according to the above-mentioned criteria. As already mentioned, polymer component b as a pure substance is not anaerobically degraded at all.

The biodegradable polyester mixtures mentioned at the outset are suitable for production of films and film strips for meshes and fabrics, tubular films, chill roll films with or without alignment in a further process step, with or without metallization or SiOx coating. With regard to the degradation characteristics, layer thicknesses of the films of 5 to 45 μm and especially of 10 to 30 μm are advantageous.

More particularly, the films comprising polymer components a) and b) are suitable for tubular films and stretch films. Possible applications here are basal fold bags, lateral seam bags, carrier bags with a hole grip, shrink labels or vest-type carrier bags, inliners, heavy-duty sacks, freezer bags, composting bags, agricultural films (mulch films), film bags for packaging of foods, peelable closure film—transparent or opaque—weldable closure film—transparent or opaque, sausage skin, salad film, freshness retention film (stretch film) for fruit and vegetables, meat and fish, stretch film for wrapping of pallets, film for nets, packaging films for snacks, chocolate bars and muesli bars, peelable lid films for dairy packaging (yoghurt, cream, etc.), fruit and vegetables, semirigid packaging, for example for smoked sausage and cheese.

Due to their barrier properties with respect to oxygen and aromas, which are excellent for biodegradable films, the films mentioned are uniquely suitable for packaging of meat, poultry, meat products, processed meat, sausages, smoked sausage, seafood, fish, crab meat, cheese, cheese products, desserts, pies, for example with meat, fish, poultry, tomato filling, pastes and bread spreads; bread, cakes, other bakery products; fruit, fruit juices, vegetables, tomato puree, salads; animal food; pharmaceutical products; coffee, coffee-like products; milk powder or cocoa powder, coffee whitener, baby food; dried foods; jams and jellies; bread spreads, chocolate cream; ready meals. For further information see reference in “Food Processing Handbook”, James G. Brennan, Wiley-VCH, 2005.

The films additionally have very good adhesion properties. As a result, they are of excellent suitability for coating of paper, for example for paper cups and paper plates. For the production thereof, both extrusion coating and lamination processes are suitable. A combination of these processes, or coating by spraying, with a coating bar or by immersion, is also conceivable.

In many countries, biomass, comprising biowaste, green waste, out of date and inedible food and drink, peelings, stalks etc. from what is called domestic waste, and also refuse, residues from the growing of foods and in the production of foods, are disposed of at refuse tips. In the course of rotting at the tips, not inconsiderable amounts of methane, a harmful greenhouse gas, pass unhindered into the atmosphere. Incineration of the biomass is also not a good alternative due to the high water content thereof and the associated poor energy balance in the course of incineration. The disposal of the biomass in composting plants and especially biogas plants (additional recovery of biogas, which can be used as an energy source) constitutes the best solution in terms of overall environmental balance. To date, the biomass has often been collected by means of paper bags or newspaper, which soak through easily, or hygienic and breathable packaging was used, for example refuse bags or packaging for foods, but these cannot be degraded in biogas plants under the anaerobic conditions which exist therein.

With the present polymer mixtures, it is for the first time possible to provide packaging (for foods, and also waste bags for biowaste) which enables disposal together with the biomass collected therein in a biogas plant. In municipalities having facilities for disposal in a biogas plant, the following process constitutes an alternative of very great interest:

A process for disposing of biomass in a biogas plant, in which in a first step the biomass is collected or dispensed in a package comprising polymer mixtures of the composition:
a) 25 to 95% by weight of a polyhydroxyalkanoate selected from the group consisting of poly-4-hydroxybutyrates, poly-3-hydroxybutyrates, poly(3-hydroxybutyrate-co-3-hydroxyvalerates), poly(3-hydroxybutyrate-co-3-hydroxyhexanoates) and poly(3-hydroxybutyrate-co-4-hydroxybutyrates) and
b) 5 to 75% by weight of an aliphatic polyester comprising:
   i) 65 to 95 mol %, based on components i to ii, of one or more C₅-C₃₆-dicarboxylic acid derivatives or C₅-C₃₆-dicarboxylic acids;
   ii) 35 to 5 mol %, based on components i to ii, of a terephthalic acid derivative or of a terephthalic acid;
   iii) 98 to 100 mol %, based on components i to ii, of a C₂-C₆-alkylenediol or C₂-C₆-oxyalkylenediol;
   iv) 0 to 2% by weight, based on components i to iii, of at least one polyfunctional compound comprising at least two isocyanate, isocyanurate, oxazoline or epoxide groups or at least three alcohol or carboxylic acid groups;
      in a second step the biomass in this package is collected by a waste management company, and in a third step the biomass in this package is sent to anaerobic fermentation in a biogas plant.

### Performance Testing:

The molecular weights Mn and Mw of the semiaromatic polyesters were determined to DIN 55672-1. Eluent: hexafluoroisopropanol (HFIP)+0.05% by weight trifluoroacetic acid Ka salt; the calibration was effected with narrow-distribution polymethyl methacrylate standards. The viscosity numbers were determined to DIN 53728 Part 3, Jan. 3, 1985, capillary viscometry. An Ubbelohde M-II microviscometer was used. The solvent used was the mixture: phenol/o-dichlorobenzene in a weight ratio of 50/50.

Modulus of elasticity and elongation at break were determined by means of a tensile test to ISO 527-3: 2003.

Tear propagation resistance was determined by an Elmendorf test to EN ISO 6383-2:2004 on specimens with constant radius (tear length 43 mm).

In a puncture resistance test, the maximum force and the puncture energy of the polyesters were measured:

The test machine used is a Zwick 1120 equipped with a spherical punch with a diameter of 2.5 mm. The sample, a circular piece of the film to be tested, was clamped perpendicularly with respect to the test punch, and this punch was moved at a constant test velocity of 50 mm/min through the plane clamped by the clamping device. Force and elongation were recorded during the test and were used to determine penetration energy.

The anaerobic digestion rates of the biodegradable polyester mixtures and of the mixtures produced for comparison were determined as follows:

The test setup and the procedure were appropriate to the corresponding method “4.1.1 Bestimmung der Biogas- and Methanausbeute in Gärtests” [Determination of the biogas and methane yields in fermentation tests] from the VDLUFA Methodenbuch VII (Umweltanalytik). The reaction vessels (fermenters) used for the determination of the biogas formation potential (anaerobic digestion) were glass vessels with a capacity of 5 l, which could be sealed gas-tight with a butyl septum and a screwtop lid. The process temperature was kept constant by means of a water bath and thermostat, in accordance with the experimental conditions (mesophilic: 38±1° C.; thermophilic: 55±1° C.). The test mixtures were mixed discontinuously, once per day.

The fermenter contents used were seed material which originated from measurements of biogas yields in a batch process and had been prepared under defined conditions (to VDI 4630). In a departure from the selected specifications (VDI 4630), however, the material had an elevated dry matter content (DM) of approx. 4.5% (w/v) and an elevated content of organic dry matter (oDM) of approx. 50% (w/w). The microorganisms present, prior to commencement of the experiment, were conditioned under anaerobic conditions without supply of substrate for a period of 5 weeks. In preparation, the fermenters were charged with 4.5 l of conditioned seed material, 30 g of the appropriate test substance were added (corresponds to a ratio of seed material oDM to test substance oDM of 3.375:1 (v/w)), the fermenter was sealed gas-tight and the gas phase of the fermenter was replaced with nitrogen. On commencement of the experiment, the biogas formed was collected in a gas collection bag, which was connected via gas-tight hose connections to the gas space of the fermenter. The volume of biogas formed was measured discontinuously; the gas composition was determined by IR measurement (CH₄, CO₂, O₂) and by means of electrochemical sensors (H₂S) in a gas chromatograph.

### Feedstocks:

### Polymer Component a (Polyhydroxyalkanoate)

### Polyester A1

Poly-3-hydroxybutyrate from PHB-Isa (trade name Biocycle 1000).

### Polymer Component b (Aliphatic-Aromatic Polyester)

### Polyester B1 (Comparative)

Polybutylene terephthalate adipate (adipic acid:terephthalic acid=50:50).

A 2 l four-neck flask is charged with 398.1 g of dimethyl terephthalate (50 mol %), 480.3 g of 1,4-butanediol (130 mol %) and 0.92 g of tetrabutyl orthotitanate (TBOT), which are heated under a nitrogen atmosphere to internal temperature 190° C. and melted. The melt is stirred constantly. Methanol is eliminated and distilled off. After approx. 90 min, 299.6 g of adipic acid (50 mol %) are added to the melt. The temperature is increased cautiously to 200° C. and the water formed is distilled off. Once no further water is being distilled over, the temperature is reduced to 190° C. A further 0.92 g of tetrabutyl orthotitanate (TBOT) is added. Vacuum is applied to the apparatus and the temperature is increased stepwise to 220° C. In the course of this, the excess 1,4-butanediol is distilled off. On attainment of the target final viscosity, the vacuum is broken and the polyester is poured onto a Teflon film.

The polyester B1 thus obtained has a viscosity number of 84 ml/g and a melting point of approx. 129° C. The molecular weight (Mn) was 16 500, the molecular weight (Mw) 40 500.

### Polyester B2 (Comparative)

Polybutylene terephthalate adipate (adipic acid:terephthalic acid=60:40), prepared analogously to polyester B1

M.p.: 104° C.

### Polyester B3

Polybutylene terephthalate adipate (adipic acid:terephthalic acid=70:30), prepared analogously to polyester B1

M.p.: 73° C.

### Polyester B4

Polybutylene terephthalate adipate (adipic acid:terephthalic acid=80:20), prepared analogously to polyester B1

M.p.: 35° C.

### Polyester B5

Polybutylene terephthalate adipate (adipic acid:terephthalic acid=90:10), prepared analogously to polyester B1

M.p.: 44° C.; 50° C.

### Polyester B6

Polybutylene adipate (adipic acid 100), prepared analogously to polyester B1

M.p.: 60° C.

### EXAMPLES

### Comparative Examples 1, 2, 6 to 8 and Examples 3 to 5

The proportions of PHB A1 and polyesters B1 to B6 specified in table 1 were compounded in an FTS 16 co-rotatory twin-screw extruder (constructed in-house) (l/d=25) at a melt temperature (head zone) of approx. 170° C., a speed of 200 min⁻¹ and a throughput of 1.5 kg/h, discharged as extrudate pellets.

All samples listed in table 1 (comparative examples 1, 2, 6 to 8 and examples 3 to 5), for the anaerobic digestion tests, were comminuted to powder using a turbo mill (from Pallmann; PPL 18).

For random powder samples, the particle size distribution was determined with a Malvern Mastersizer 2000. For example, for the mixture C1 (50% polybutylene terephthalate adipate (adipic acid:terephthalic acid=50:50) and 50% PHB), the measured characteristics (d_{10/50/90}) were 88 μm/245 μm/538 μm. For all samples analyzed, the d₉₀ value was below 1000 μm. This means that 90% by volume of the powder has a particle size less than 1000 μm.

TABLE 1

| Example | C1 | C2 | 3 | 4 | 5 | C6 | C7 | C8 |
|---|---|---|---|---|---|---|---|---|
| Comp. A1 | 50 | 50 | 50 | 50 | 50 | 50 | — | 100 |
| [% by wt.] | | | | | | | | |
| Comp. B1 | 50 | | | | | | | |
| [% by wt.] | | | | | | | | |
| Comp. B2 | | 50 | | | | | | |
| [% by wt.] | | | | | | | | |
| Comp. B3 | | | 50 | | | | | |
| [% by wt.] | | | | | | | | |
| Comp. B4 | | | | 50 | | | | |
| [% by wt.] | | | | | | | | |
| Comp. B5 | | | | | 50 | | | |
| [% by wt.] | | | | | | | | |
| Comp. B6 | | | | | | 50 | 100 | |
| [% by wt.] | | | | | | | | |
| Biogas | 484 | 520 | 559 | 653 | 642 | 536 | 18 | 1004 |
| evol.* | | | | | | | | |
| [l/kg] | | | | | | | | |
| Biogas | 495 | 553 | 620 | 782 | 713 | 583 | 22 | 1005 |
| evol.** | | | | | | | | |
| [l/kg] | | | | | | | | |
| Biogas | 499 | 571 | 645 | 832 | 733 | 605 | 19 | 1005 |
| evol.*** | | | | | | | | |
| [l/kg] | | | | | | | | |
| Methane | 58.3 | 58.7 | 58.6 | 58.7 | 58.8 | 58.9 | — | 56.2 |
| content in | | | | | | | | |
| biogas at | | | | | | | | |
| end of | | | | | | | | |
| experiment | | | | | | | | |
| [%] | | | | | | | | |

*Biogas evolution after 21 days
**Biogas evolution after 42 days
***Biogas evolution after 56 days

The data depicted in table 1 show the biogas yields (CO₂+CH₄) of the various polymer mixtures after an incubation period of 21, 42 and 56 days under mesophilic conditions at 38° C. In comparative experiments C7 and C8, two individual components of the polymer mixtures were used in the degradation test. For component A1 (PHB, see C8), with a biogas evolution of 1005 l/kg after only 14 days, full anaerobic biodegradation is observed. Component B6 (polybutylene adipate, C7) shows no significant degradation even after 56 days.

In mixtures with poly-3-hydroxybutyrate, particularly under mesophilic conditions in examples 3 to 5, biogas production significantly above the theoretically expected value (50% PHB in the mixture corresponds to max. 503 l/kg of biogas) is found (3: +28%; 4: +65%; 5: +45%). This synergistic effect is surprising. The biodegradability of the polymer mixture which was used in experiments C1, C2 and C6 is much lower within the time interval considered.

1-6. (canceled)

7. A process for complete anaerobic digestion of polymer mixtures of the composition:
a) 25 to 95% by weight of a polyhydroxyalkanoate selected from the group consisting of poly-4-hydroxybutyrates, poly-3-hydroxybutyrates, poly(3-hydroxybutyrate-co-3-hydroxyvalerates), poly(3-hydroxybutyrate-co-3-hydroxyhexanoates) and poly(3-hydroxybutyrate-co-4-hydroxybutyrates), and
b) 5 to 75% by weight of an aliphatic-aromatic polyester comprising:
i) 65 to 95 mol %, based on components i to ii, of one or more C₅-C₃₆-dicarboxylic acid derivatives or C₅-C₃₆-dicarboxylic acids;
ii) 35 to 5 mol %, based on components i to ii, of a terephthalic acid derivative or of a terephthalic acid;
iii) 98 to 100 mol %, based on components i to ii, of an C₂-C₈-alkylenediol or C₂C₆-oxyalkylenediol;
iv) 0 to 2% by weight, based on the polymer obtainable from components i to iii, of at least one polyfunctional compound comprising at least two isocyanate, isocyanurate, oxazoline or epoxide groups or at least three alcohol or carboxylic acid groups.

8. The process according to claim 7, wherein the polymer mixture comprises:
a) 25 to 50% by weight of the polyhydroxyalkanoate, and
b) 50 to 75% by weight of the aliphatic-aromatic polyester.

9. The process according to claim 7, wherein the polyhydroxyalkanoate (polymer component a)) used is a poly(3-hydroxybutyrate-co-3-hydroxyvalerate), poly(3-hydroxybutyrate-co-3-hydroxyhexanoate) or a poly(3-hydroxybutyrate-co-4-hydroxybutyrate).

10. The process according to claim 7, wherein 5 to 50% by weight of thermoplasticized starch, based on the total weight of components a to c, are added to the polymer mixture as a further component c).

11. A process for complete anaerobic digestion of packaging for biological material, comprising polymer mixtures of the composition:
a) 25 to 95% by weight of a polyhydroxyalkanoate selected from the group consisting of poly-4-hydroxybutyrates, poly-3-hydroxybutyrates, poly(3-hydroxybutyrate-co-3-hydroxyvalerates), poly(3-hydroxybutyrate-co-3-hydroxyhexanoates) and poly(3-hydroxybutyrate-co-4-hydroxybutyrates), and
b) 5 to 75% by weight of an aliphatic-aromatic polyester comprising:
i) 65 to 95 mol %, based on components i to ii, of one or more C₅-C₃₆-dicarboxylic acid derivatives or C₅-C₃₆-dicarboxylic acids;
ii) 35 to 5 mol %, based on components i to ii, of a terephthalic acid derivative or of a terephthalic acid;
iii) 98 to 100 mol %, based on components i to ii, of an C₂-C₈-alkylenediol or C₂-C₆-oxyalkylenediol;
iv) 0 to 2% by weight, based on the polymer obtainable from components i to iii, of at least one polyfunctional compound comprising at least two isocyanate, isocyanurate, oxazoline or epoxide groups or at least three alcohol or carboxylic acid groups.

12. A process for disposing of biomass in a biogas plant which comprises in which in a first step the biomass is collected or dispensed in a package comprising polymer mixtures of the composition:
a) 25 to 95% by weight of a polyhydroxyalkanoate selected from the group consisting of poly-4-hydroxybutyrates, poly-3-hydroxybutyrates, poly(3-hydroxybutyrate-co-3-hydroxyvalerates), poly(3-hydroxybutyrate-co-3-hydroxyhexanoates) and poly(3-hydroxybutyrate-co-4-hydroxybutyrates) and
b) 5 to 75% by weight of an aliphatic polyester comprising:
i) 65 to 95 mol %, based on components i to ii, of one or more C₅-C₃₆-dicarboxylic acid derivatives or C₅-C₃₆-dicarboxylic acids;
ii) 35 to 5 mol %, based on components i to ii, of a terephthalic acid derivative or of a terephthalic acid;
iii) 98 to 100 mol %, based on components i to ii, of a C₂-C₈-alkylenediol or C₂-C₆-oxyalkylenediol;
iv) 0 to 2% by weight, based on the polymer obtainable from components i to iii, of at least one polyfunctional compound comprising at least two isocyanate, isocyanurate, oxazoline or epoxide groups or at least three alcohol or carboxylic acid groups;
in a second step the biomass in this package is collected by a waste management company, and in a third step the biomass in this package is sent to anaerobic fermentation in a biogas plant.
